(19) United States
(12) Patent Application Publication
(10) Pub. No.: US 2006/0002967 A1
(43) Pub. Date: Jan. 5, 2006

(54) Resorbable implant with lubricious coating

(76) Inventors: Thomas L. Smestad, Palo Alto, CA (US); John Ferdinand Dichiara, New York, NY (US)

Correspondence Address: June E. Cohan;Shaw Pittman LLP, 1650 Tysons Boulevard, McLean, VA, 22102, US

(21) Appl. No.: 10/880,559

(22) Filed: Jul. 1, 2004

(51) Int. Cl. A61K 31/737; A61F 2/00

(52) U.S. Cl. 424/423; 514/54

(57) ABSTRACT

A device for surgical implantation that has a lubricious coating disposed on a polymeric matrix. The polymeric matrix establishes a biocompatible and at least partially bioresorbable scaffold adapted for ingrowth of native tissue. The lubricious coating is biocompatible and bioresorbable, and acts to lubricate the matrix and decrease friction on its surface. The lubricious coating may be discrete or integrated, and may be disposed on part or the whole of the matrix surface.

### FIELD OF THE INVENTION

The present invention is in the field of implantable medical devices, and more particularly is directed to bioresorbable implants such as meniscal implants, and methods for their fabrication.

### BACKGROUND OF THE INVENTION

Implants are widely used for reconstruction of damaged tissues. Such implants include dental implants, hip and knee implants, plates and pins for broken bones, and other devices. Some implants are successful in reducing the suffering and disabilities associated with tissue damage, however, many fail to perform long-term functions because the implant material deteriorates within the human body. Joint implants, particularly implants designed for free-moving synovial joints, may also cause problems in that the implant may damage natural tissue or become dislodged due to shear stress and other mechanical forces. A particular problem arises with implants in the knee joint, because the joint is weight-bearing as well as free-moving.

The medial and lateral menisci are a pair of crescent-shaped cartilaginous structures in the knee joint that are anatomically located between the femoral condyles and tibial plateau. FIG. 1 shows a diagrammatic representation of the normal positioning of medial meniscus 7 and lateral meniscus 8 in the human knee joint 3 between the femur 2 and tibia 4. These menisci, when compressed between the femur 2 and tibia 4, become tough except at their points of attachment. FIG. 2 shows the in vivo structure of medial meniscus 7 and lateral meniscus 8 in the knee joint 3. The menisci conform to the shapes of the surfaces between which they are positioned, resulting in two distinct in vivo forms. For example, the medial meniscus 7 has a relatively open crescent shape, while the lateral meniscus 8 has a relatively closed crescent shape.

Together, the menisci act to stabilize the joint, distribute force and load, and lubricate the area of contact between the tibia and femur. Each meniscus has a thickness of about 7 to 8 mm at the periphery and gradually tapers to a thin tip at the inner margin, forming a slightly concave triangle in cross-section. The major portion of the meniscal tissue is avascular except the peripheral rim, which comprises about 10% to 30% of the total width of the structure and is nourished by the peripheral vasculature. The avascular meniscal tissue is composed of fibrochondrocytes surrounded by an abundant extracellular matrix and water (about 70% of tissue weight) where the nutrients are provided presumably through physicochemical processes. Collagen (primarily type I) accounts for the majority of the matrix material, amounting to about 75% by weight of the dry tissue, and the collagen fibers are oriented primarily in the circumferential direction.

The menisci are primarily designed to withstand compressive forces and to share load bearing with the articular cartilage, and can be torn by strong shear forces that result from rapid rotational movement in the joint. Damage to the menisci may also occur chronically as part of degenerative changes within the knee. Common athletic injuries to the knee may result in tearing the meniscus tissue. Damage of torn tissue in the peripheral vascular rim can be accomplished arthroscopically, with sutures or similar techniques, and the wound usually heals with the return of normal meniscus function. Where the injured site is in the avascular region, however, repair of the damaged tissue is often inadequate or impossible, and partial or total removal of the damaged meniscus tissue is often indicated. Without the menisci, stress concentration occurs in the knee in conjunction with abnormal joint mechanics, and premature development of arthritic changes occurs.

Treatment of injured or diseased menisci has generally been both by surgical repair and by excision. With excision, regeneration of meniscal tissue may occur. Additionally, it is known that meniscal fibrochondrocytes have the ability to migrate into a defect filled with a fibrin clot and form tissue apparently similar to normal meniscal fibrocartilage. When an adequate matrix scaffold is present within a meniscal defect, such meniscal fibrocartilage may be formed. Meniscal tissue is also capable of self-repair when exposed to bleeding tissues, and additionally, it is also known in the art that meniscal cells in tissue culture are capable of cell division and matrix synthesis. Replacement of an injured meniscus in an otherwise healthy joint may prevent arthritic changes and may stabilize the joint. In diseased joints, replacement of the meniscus may reduce the progression of the disease process, and may provide pain relief. Allografting or meniscal transplantation is one method of replacement which has been executed both in dogs and in humans. However, this approach has been only partially successful over the long term due to the host's immunologic response to the graft, to failures in the cryopreservation process, and to failures of the attachment sites.

One prior art replacement approach has been to replace menisci with prostheses composed of permanent artificial materials. The use of such materials is believed to minimize the possibility of an immunological response, permit construction of a structure which can withstand the high and repeated loads which are encountered in the knee joint, and alter the joint mechanics in beneficial ways that biological materials would not tolerate. Replacement of meniscal tissue with these permanent artificial structures generally has been unsuccessful, however, principally because the opposing articular cartilage of human and animal joints is fragile and easily damaged by abrasive interfaces. Additionally, joint forces are multiples of body weight which, in the case of the knee and hip, are typically encountered over a million cycles per year. Permanent artificial menisci have not been composed of materials having natural meniscal properties, nor have they been able to be positioned securely enough to withstand the routine forces bearing on the joint.

Another, more successful approach has been to replace menisci with a biocompatible and bioresorbable structure for implantation into the knee joint which assumes the form and role of a meniscus. Stone (U.S. Pat. Nos. 5,007,934, 5,116,374, and 5,158,574) and Li et al. (U.S. Pat. Nos. 5,681,353, 5,735,903, and 6,042,610) describe prosthetic menisci comprising biocompatible and bioresorbable fibers, such as natural polymers, and methods for fabricating such prosthetic menisci. In addition, Stone describes methods of regenerating meniscal tissue by implanting the resorbable prosthetic meniscus into a human knee, such as by sewing the implant into the host meniscal rim.

The resorbable meniscus also acts as a scaffold for regenerating meniscal tissue by encouraging the ingrowth of meniscal tissue and meniscal fibrochondrocytes, resulting in a composite of the host meniscus and the implanted device serving the role of a natural meniscus. A resorbable meniscus has been shown to provide potential long-term benefits to human patients. Like most joint implants, however, the device requires the recipient to undergo a period of rehabilitation after implantation. Some of this rehabilitation time results from the resorbable implant being susceptible to mechanical damage and dislodgement before significant tissue ingrowth has occurred. Post-implantation, repetitive shear stresses, particularly at the inner margin of the implant, may damage the implant prior to complete integration of the newly regenerated tissue with the host meniscus. Long rehabilitation times also introduce risks of tearing and suture pull-out.

Thus, a need exists for an implant having a surface that is less susceptible to mechanical damage and dislodgement, and more resistant to repetitive shear forces that result in tearing and suture pull-out.

### SUMMARY OF THE INVENTION

The present invention provides a device for surgical implantation to replace damaged tissue in a joint, particularly the menisci of the knees, that has a lubricious coating on a polymeric matrix. This lubricious coating is disposed on part or all of the matrix surface, and is preferably disposed on one or more articulating surfaces of the matrix. The polymeric matrix establishes a biocompatible and at least partially bioresorbable scaffold adapted for ingrowth of native tissue. The lubricious coating serves to decrease friction and resulting shear forces on the surface of the scaffold, thereby enhancing the implant's resistance to shear forces and dislodgement.

Additional advantages and features of the present invention will be apparent from the following drawings, detailed description and examples which illustrate preferred embodiments of the invention.

### BRIEF DESCRIPTION OF THE DRAWINGS

FIG. 1 shows a simplified diagrammatic representation of a human knee joint, with menisci in native positioning.

FIG. 2 is a diagrammatic representation of a cut-away view of the knee joint showing the medial and lateral menisci as they are positioned in vivo over the medial and lateral condyles.

FIG. 3 shows a perspective view of an exemplary prosthetic meniscus implant in accordance with the present invention.

FIG. 4 shows a sectional view along line 5-5 of the exemplary prosthetic meniscus of FIG. 3 prior to being coated.

FIGS. 5a and 5b show top and cross-sectional schematic views of an exemplary medial meniscus implant, after it is partially coated on a superior articulating surface.

FIGS. 6a and 6b show top and cross-sectional schematic views of an exemplary medial meniscus implant, after it is completely coated on a superior articulating surface.

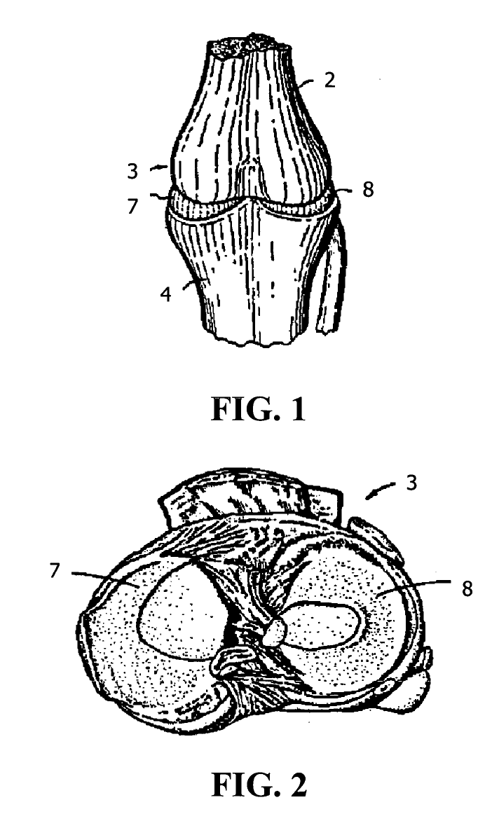

### DETAILED DESCRIPTION OF THE INVENTION

Reference will now be made in detail to the presently preferred embodiments of the invention, which, together with the following examples, serve to explain the principles of the invention. These embodiments are described in sufficient detail to enable those skilled in the art to practice the invention, and it is to be understood that other embodiments may be utilized, and that structural, chemical, and biological changes may be made without departing from the spirit and scope of the present invention.

The present invention is a biocompatible implant comprising a polymeric matrix, and a lubricious coating on the surface of at least part of the matrix. The implant is designed for use in the skeletomuscular system, and can be a cartilage implant, a ligament implant, a tendon implant, an interpositional joint spacer, a spinal disc implant, a meniscus implant, or a bone implant. In one embodiment, the implant is a meniscus implant. Biocompatible implants can also be designed for implantation in or near a joint, such as, but not limited to, any synovial joint, such as the knee, hip, shoulder (acromioclavicular, stemoclavicular) and wrist joints. Suitable implant hosts include, but are not limited to, humans and animals, including dogs, cats, horses, cows, sheep, birds, fish, or reptiles, and preferably are mammals, and even more preferably are humans.

The meniscus implant of the present invention can be a prosthetic meniscus intended to wholly replace a natural meniscus, or the implant can be a meniscal augmentation device, which is meant to replace part of the natural meniscus. For example, a meniscal augmentation device can be implanted into a defect, such as a segmental defect, of a meniscus in a host. A segmental meniscus defect typically encompasses a tear or lesion (including radial, horizontal, bucket handle, and complex tears) in less than the entire meniscus, resulting in partial resection of the meniscus. Upon implantation into a segmental defect of a meniscus, the composite formed by the partial meniscus and the implant has an in vivo outer surface contour substantially the same as a whole natural meniscus without a segmental defect.

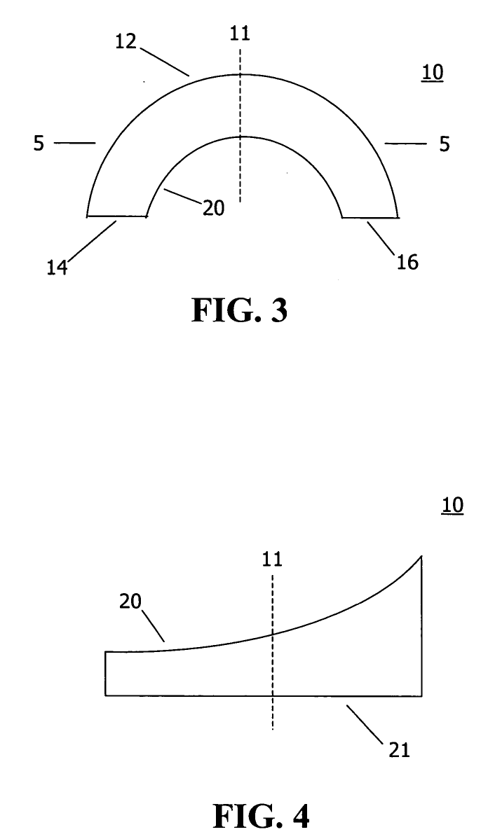

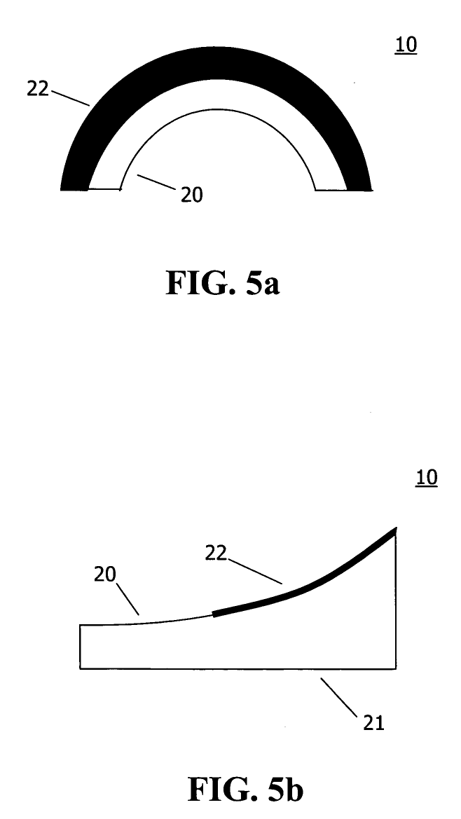

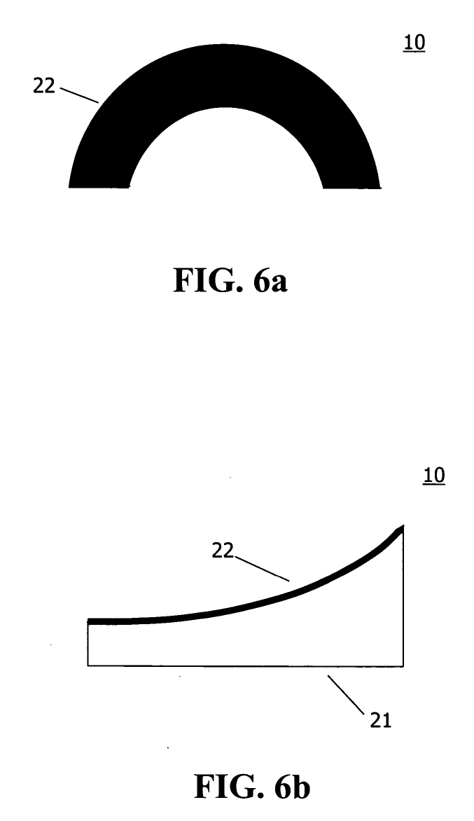

An exemplary meniscal implant is shown in FIG. 3. The meniscal implant 10 is generally wedge-shaped, and extends circumferentially or laterally at least in part around a central axis 11. In a preferred form, the meniscus 10 has the shape of a semicircular-shaped wedge, extending circumferentially around the axis 11, and comprising a relatively wide central region 12 between two narrow distal regions 14 and 16. FIG. 4 is a sectional view taken along line 5-5 of the meniscus shown in FIG. 3. FIG. 4 shows the meniscus 10 prior to coating, in which the superior articulating surface 20 (the surface that will face the femoral condyles upon implantation) has not been coated. The inferior articulating surface 21 (the surface that will face the tibial plateau upon implantation) is also shown in FIGS. 4-6. FIG. 5 shows a medial meniscus implant 10 after superior articulating surface 20 has been partially coated with lubricious coating 22, and FIG. 6 shows a medial meniscus implant 10 of a different embodiment, after articulating surface 20 has been completely coated with lubricious coating 22.

The Polymeric Matrix

The polymeric matrix of the implant is biocompatible and at least partially bioresorbable. A biocompatible material is a material that is of specific strength, permeability, durability, etc., and is able to perform in a specific application within a host without causing undesirable biological reactions such as an acute inflammatory reaction. A bioresorbable material is one that can degrade, e.g., by proteolysis, to lower molecular weight fragments which can be used by the host or are readily removed and or further degraded by the host. Examples of such biocompatible and bioresorbable materials include, but are not limited to, various types of collagens, polysaccharides, other proteins including fibrin or elastin, and collagen-based materials (i.e., a material that includes or is derived from collagen) such as a collagen-heparin composite, a collagen-growth factor composite and a collagen-cell composite.

The polymeric matrix can be a synthetic polymer-based matrix, or a biopolymer-based matrix. In a preferred embodiment, the matrix comprises a natural material, preferably natural polymers, which can provide lubrication as well as mechanical strength. Suitable biopolymeric materials include, but are not limited to, proteins (e.g., collagen, gelatin, fibrin, elastin, or silk) and polysaccharides (e.g., hyaluronic acid, alginic acid, cellulose, chitin). In a preferred embodiment, the polymeric matrix is made of collagen-based material, and preferably of Type I collagen fibers. Collagen can be isolated from human or animal tissues, such as tendon, skin, bone, or ligament, produced by genetic engineering (e.g., collagen marketed by Fibrogen, Inc., Palo Alto, Calif.), or synthesized by fibroblasts in vitro (e.g., collagen prepared by Advanced Tissue Sciences, La Jolla, Calif.). The fibers can be randomly oriented throughout the matrix, or can assume a substantially circumferentially extending or substantially radially extending orientation throughout the matrix. The density of the fibers can be uniform or non-uniform throughout the matrix, and in the non-uniform configuration, relatively high densities of fibers can be established at anticipated points of high stress.

The polymeric matrix typically has the form of a dry, porous volume matrix, a portion of which can be crosslinked. A porous matrix encourages ingrowth of meniscal fibrochondrocytes, endothelial cells, fibroblasts, and other cells that normally occupy the extracellular matrix as well as synthesize and deposit extracellular matrix components. Certain characteristics of the matrix, such as the rate of tissue ingrowth, the rates of resorption of the scaffold and of the individual fibers, and the stability of the matrix shape in vivo, are affected by the matrix density, and the degree of crosslinking in the matrix.

For example, in order to encourage ingrowth while at the same time preserving mechanical strength and cushioning ability, the density of the matrix can be manipulated. If a relatively great intrafibrillary and interfibrillary space is desired to encourage tissue growth into the matrix, the preferred matrix density is in the range from about 0.07 to about 0.15 g/cm³. If a relatively small intrafibrillary and interfibrillary space is desired to provide mechanical support for the knee joint and improve cushioning, the preferred matrix density is in the range from about 0.15 to about 0.50 g/cm³. In a preferred embodiment, the matrix has a density of about 0.10 to about 0.25 g/cm³ with an intrafibrillary and interfibrillary space of about 8 cm³/cm³ matrix to about 9 cm³/cm³ matrix, which offers an ideal environment for ingrowth of meniscal fibrochondrocytes as well as other cells while maintaining sufficient mechanical strength to support natural meniscal load forces.

In a preferred embodiment, the polymeric matrix has a pore size substantially in the range of 10-5000 microns, in a more preferred embodiment the pore size is substantially in the range of 50-1000 microns, and in an even more preferred embodiment the pore size is substantially in the range of 50-500 microns. For a load-bearing implant such as a meniscal implant, the pore sizes will tend to be smaller, so that the implant has sufficient mechanical strength to support natural load forces. For a non-weight-bearing implant such as an interpositional joint spacer or a ligament implant, the pore size may be larger.

In a preferred embodiment, the matrix also includes glycosaminoglycan molecules (GAGs) interspersed throughout the fibers. These GAGs provide lubrication and crosslinks for the matrix. Examples of GAGs that can be used in the present invention include, but are not limited to, chondroitin 4-sulfate, chondroitin 6-sulfate, keratan sulfate, dermatan sulfate, heparin, heparan sulfate, hyaluronic acid, and combinations thereof as components of the polymeric matrix. In a preferred embodiment a combination of chondroitin sulfate and hyaluronic acid is used as a component of the polymeric matrix. The GAGs can be uniformly dispersed throughout the polymeric matrix as individual molecules, or they can be present in varying amounts in different regions of the polymeric matrix. In a particular preferred embodiment, the polymeric matrix can be composed of about 75-100% natural fibers and about 0-25% GAGs by dry weight. These proportions can be constant or variable throughout the polymeric matrix.

In one embodiment, a polymeric matrix of the present invention has a composition, by dry weight, of: about 50% or greater natural fibers and about 50% or less GAGs; about 55% or greater natural fibers and about 45% or less GAGs; about 60% or greater natural fibers and about 40% or less GAGs; about 65% or greater natural fibers and about 35% or less GAGs; about 70% or greater natural fibers and about 30% or less GAGs; about 75% or greater natural fibers and about 25% or less GAGs; about 80% or greater natural fibers and about 20% or less GAGs; about 85% or greater natural fibers and about 15% or less GAGs; about 90% or greater natural fibers and about 10% or less GAGs; about 95% or greater natural fibers and about 5% or less GAGs; or about 98% or greater natural fibers and about 2% or less GAGs.

In another embodiment, a polymeric matrix of the present invention has a composition, by dry weight, of: about 50%-99% natural fibers and about 1%-50% GAGs; about 60%-99% natural fibers and about 1%-40% GAGs; about 65%-99% natural fibers and about 1%-35% GAGs; about 70%-99% natural fibers and about 1%-30% GAGs; about 75%-99% natural fibers and about 1%-25% GAGs; about 80%-99% natural fibers and about 1%-20% GAGs; about 85%-99% natural fibers and about 1%-15% GAGs; or about 90%-99% natural fibers and about 1%-10% GAGs.

In a different embodiment, a matrix of the present invention has a composition, by dry weight, of: about 50%-75% natural fibers and about 25%-50% GAGs; about 60%-75% natural fibers and about 25%-40% GAGs; about 65%-75% natural fibers and about 25%-35% GAGs; about 70%-95% natural fibers and about 5%-30% GAGs; about 70%-85% natural fibers and about 15%-30% GAGs; about 70%-80% natural fibers and about 20%-30% GAGs; about 75%-95% natural fibers and about 5%-25% GAGs; about 75%-85% natural fibers and about 15%-25% GAGs; about 80%-95% natural fibers and about 5%-20% GAGs; about 80%-90% natural fibers and about 10%-20% GAGs; or about 85%-95% natural fibers and about 5%-15% GAGs.

The temporary stability of the matrix shape in vivo, and the rate of resorption of the fibers (and GAGs if the matrix contains GAGs), are both attributed to crosslinks between at least a portion of the fibers. In addition, GAGs can directly participate in the formation of covalent crosslinks with the fibers, or can interact mechanically with the fibers by entanglement to form stable fiber-GAG complexes. The matrix constituents can be cross-linked either by chemical reagents or by other means such as UV light.

In a preferred embodiment, the polymeric matrix is a porous dry volume matrix of Type I collagen fibers interspersed with glycosaminoglycan molecules, where the collagen fibers are present at a concentration of about 65%-99% by dry weight, and the glycosaminoglycan molecules are present at a concentration of about 1%-35% by dry weight. At least a portion of the glycosaminoglycan molecules provide crosslinks between the collagen fibers, where the crosslinks are dispersed substantially nonuniformly throughout the matrix. This preferred matrix has a pore size substantially in the range of 50-500 microns, and the matrix establishes an at least partially bioresorbable scaffold adapted for ingrowth of meniscal fibrochondrocytes.

In another preferred embodiment, the polymeric matrix is a dry porous matrix of biocompatible bioresorbable fibers that are selected from the group consisting of natural polymers and analogs and combinations thereof. This matrix has a pore size in the approximate range of greater than 50 microns to less than about 500 microns, and it establishes an at least partially bioresorbable scaffold adapted for ingrowth of meniscal fibrochondrocytes, where the scaffold and the ingrown meniscal fibrochondrocytes support natural meniscal load forces.

The Lubricious Coating

The polymeric matrix of the present invention has a coating disposed on its surface. The coating provides a lubricious outer surface for the implant and results in multiple advantages, including facilitating insertion and maneuverability of the implant during surgery, lowering the coefficient of friction for the implant, decreasing shear forces exerted on the implant and reducing damage to surfaces in the body which contact the implant.

The lubricious coating can be disposed on the entire surface of the matrix or a portion thereof, and preferably is disposed only on one or more “articulating surfaces” of the matrix, i.e., surfaces that upon implantation will rub, press or slide against a native cartilage or bone surface inside the joint. For a meniscus implant, the lubricious coating is preferably disposed on the superior articulating surface, i.e., the surface of the matrix that will face the femoral condyles upon implantation, and may optionally also be disposed on the inferior articulating surface, i.e, the surface of the matrix that will face the tibial plateau upon implantation. For an interpositional joint spacer used, for example, in the proximal interphalangeal joint of the hand, the lubricious coating is preferably disposed on the articulating surfaces, i.e., the surfaces of the matrix that will face the proximal and middle phalanges upon implantation. Coating the matrix only on the articulating surface(s) localizes the lubricious benefits of the coating, and prevents the coating from possibly interfering with tissue ingrowth into the matrix on the uncoated surfaces.

The lubricious coating of the present invention can be a discrete coating on the matrix substrate, or it can be at least partially integrated with the matrix itself. A discrete coating covers (at least in part) the polymeric matrix surface rather like the peel of an apple covers the flesh, whereas an at least partially integrated coating is more like the membrane on an orange segment in that it is difficult to cleanly separate the coating from the material which it coats.

There are various advantages and disadvantages of these different types of coatings, as will be evident to one of skill in the art. For example, a discrete coating has an advantage in that it can be specifically designed to enhance lubricity without much regard to the composition of the polymeric matrix, whereas an integrated coating (whether partially or wholly integrated) is designed to interact with the polymeric matrix to some degree, such as, for example, by crosslinking to one or more components of the matrix itself, and therefore must be chosen with an eye to the matrix composition. As another example, an integrated coating has an advantage over a discrete coating because, by nature of its being integrated, it often has greater adhesive strength than a discrete coating, and such greater adherence can be desirable under certain circumstances known to those of skill in the art.

The lubricious coating of the present invention can have one or a combination of several physical forms. For example, it can be a solid mixture, such as, e.g., a mixture of stabilizing polymers, hydrophilic polymers, additives, and possibly solvent residues blended together. Alternatively, the coating can be a solid solution, that is a mixture uniformly dispersed throughout the solid phase with homogeneity at the molecular or ionic level, or it can be a combination of dissolved and mixed components, such as a mixture of a polymer coating solution and insoluble particles in suspension. The coating can take the form of a composite, that is a structure composed of a mixture or combination of polymer and other constituents that differ in form and chemical composition and are essentially insoluble in each other. It can be referred to as a matrix of polymer in which other components are entrapped. The coating can comprise separate layers, discrete or intermingled, each of which can have any or several of these forms.

The lubricious coating is biocompatible and bioresorbable. In a preferred embodiment, the rate of resorption of the lubricious coating is designed to be greater than the rate of resorption of the polymeric matrix. This greater resorption rate results in the coating “wearing off” the surface of the implant at a faster rate than the resorption of the polymeric matrix, which has the advantage of providing ongoing lubricity to the implant, first from the coating itself, and later from tissue ingrowth in the polymeric matrix and subsequent production of natural lubricants.

In a preferred embodiment, the coating is a bioresorbable coating, and may be synthetic or natural in origin. The coating is not a biomembrane, such as a peritoneum or small intestine submucosal membrane, but it may be of biological origin. Exemplary resorbable coating materials include, but are not limited to, proteins (e.g., collagen, gelatin, fibrin, elastin, or silk) and polysaccharides (e.g., dextran, hyaluronic acid, alginic acid, cellulose, chitin), heparin and derivatives of these substances, for example cellulose derivatives such as methyl cellulose, carboxymethyl cellulose, hydroxyethyl cellulose and hydroxypropyl cellulose. In a preferred embodiment, the coating is made of collagen-based material, and preferably of Type I collagen fibers. Other suitable resorbable materials include, but are not limited to, glycosaminoglycan molecules (GAGs), including chondroitin 4-sulfate, chondroitin 6-sulfate, keratan sulfate, dermatan sulfate, heparin, heparan sulfate, hyaluronic acid, and combinations thereof. A particularly preferred resorbable coating is a solution of sodium hyaluronate, which can be in the form of a discrete coating or can be crosslinked to the polymeric matrix to form a partially or wholly integrated coating.

In another preferred embodiment, the coating is a hydrophilic polymer-based coating, i.e., it absorbs water and swells in an aqueous environment to become a hydrogel, which affords lubricity or “slip” to the coating when wet. A skilled artisan is able to select suitable hydrophilic polymers based on knowledge of the art, and by referring to resources such as the Concise Encyclopedia of Polymer Science and Engineering, Kroschwitz, ed. (Wiley 1990), and to Whitbourne (U.S. Pat. Nos. 5,001,009 and 5,525,348, which relate to hydrophilic coatings containing cellulose ester polymers). The concentration and type of the hydrophilic component in the coating is sufficient to absorb water and become lubricious when wet, while being compatible with other components in the coating, and (if integrated) the polymeric matrix.

Exemplary hydrophilic polymers include, but are not limited to, poly(N-vinyl) lactams such as polyvinyl pyrrolidone (PVP) and the like, polyethylene oxide (PEO), polyethylene glycol (PEG), polypropylene oxide (PPO), polyacrylamides, cellulosics such as methyl cellulose and the like, polyacrylic acids such as acrylic and methacrylic acids and the like, polyvinyl alcohols, and polyvinyl ethers and the like. Other hydrophilic polymers include, but are not limited to, polyethers, polyethylene glycol, polysaccharides, hydrophilic polyurethanes, polyhydroxyacrylates, polymethacrylates, and copolymers of vinyl compounds and hydroxyacrylates or acrylic acid, so long as the appropriate hydrophilicity is present. Other examples include, but are not limited to, dextran, xanthan, hydroxypropyl cellulose, methyl cellulose, polyacrylamide, and polypeptides.

The hydrophilic polymer can be of any molecular weight, but it preferably has an average molecular weight in the range of about 50,000 to 5,000,000. For an integrated coating, the molecular weight of the hydrophilic polymer has an effect on its adherence to the polymeric matrix, i.e., its degree of immobilization. It is contemplated that for hydrophilic polymers of lower molecular weights, e.g., less than about 250,000, the crosslink density between the coating polymer and the polymeric matrix will be adjusted upward accordingly. The “desired” level of crosslink density is that which will entrain and substantially restrict the mobility of the hydrophilic polymeric coating without compromising its functionality, e.g., lubricity. Crosslinking or covalent anchoring may not be appropriate in all instances, however, as restriction of the mobility of the hydrophilic polymer can adversely affect “slip” or lubricating properties of the coating. It is contemplated that such covalent anchoring of the hydrophilic polymer may be suitable used with hydrophilic polymers that have been copolymerized with reactive monomers, such as PVP/RCOOH, PVP or PVOH/anhydrides or PVP/acetamide.

Methods of Making Implants

Creation of the implant of the present invention uses manufacturing techniques known to those skilled in the art. The specific method of manufacture is not critical to the invention; any suitable manufacturing method that provides an implant having the desired characteristics of the present invention will suffice. In general, the polymeric matrix is fabricated either wholly or partially before the introduction of the coating, depending on whether or not the coating will be integrated into the polymeric matrix. For example, if an integrated coating is desired then the coating components will be introduced to the polymeric matrix components at an appropriate stage of fabrication, for example before or after a first crosslinking step, before or after lyophilization, or before or after a second crosslinking step.

The method for fabricating a polymeric matrix is known in the art (e.g., U.S. Pat. Nos. 5,007,934, 5,116,374 and 5,735,903) and is incorporated herein as if set out in full. The method generally includes placing a plurality of fibers (or fibers and GAGs, or fibers and growth factors and/or adhesion factors and/or GAGS), into a mold, lyophilizing the fibers, and contacting the fibers or the fibers and GAGs with a chemical crosslinking reagent such that the fibers or the fibers and GAGs assume the shape of the mold to obtain a dry, porous volume matrix. Alternatively, an additional crosslinking step is performed by lyophilizing the chemically crosslinked matrix and then subjecting it to dehydrothermal crosslinking procedures. Specific densities and pore sizes can be obtained in various regions of the matrix by compressing the fibers or the fibers and GAGs in the mold prior to the chemical crosslinking step. This can be accomplished by applying pressure to a specific region of the matrix with a piston of a predetermined shape.

The fibers can be placed randomly or oriented in specific directions in, for example, mold forms such as a cylindrical form. For example, the fibers can be placed in the mold in a circumferential orientation by rotating the mold as the fibers are placed therein. Alternatively, the fibers can be oriented radially in the mold by manually painting the fibers in a linear, radially directed pattern. Other components such as GAGs which can participate in the crosslinking reactions, can be mixed in with the fibers in a random or non-random fashion before the structure is subjected to various crosslinking and dehydrating procedures including various chemical and/or dehydrothermal methods. Adhesion molecules or adhesive fragments or analogs thereof, or growth factors or biologically active fragments or analogs thereof, can be incorporated into this structure during processing.

Coating of the matrix can take place at different stages during fabrication of the implant, depending on the type and placement of the coating. For example, a coating material can be applied to the matrix prior to crosslinking of the fibers, resulting in a highly integrated coating, or it can be applied to the matrix after crosslinking of the fibers, which will result in a less integrated coating. Also, the matrix can be lyophilized before or after application of the coating material, which will also affect the final nature of the coating, as is understood by those of skill in the art. The matrix can be subjected to an additional dehydrothermal crosslinking process after the coating is applied. Depending on the coating used, the coating material may be applied to the matrix by brushing, spraying, rolling, dipping, stamping or the like. Particularly discrete coatings may be placed on the matrix in a pre-formed state, and affixed by pressing, gluing, rolling, or the like. Regardless of the method used, care is taken to ensure that the pores of the matrix are not unnecessarily clogged, but that a majority remain patent and of a desired diameter.

Reagents useful as cross-linking agents include, but are not limited to, those described in, e.g., U.S. Pat. No. 6,177,514, and can interact with amino, carboxyl, orhydroxyl groups on a single molecule, on different molecules, or on fibers and GAGs, to form intramolecular crosslinks. The crosslinking reagents used to form these crosslinks include biocompatible bifunctional reagents. Useful crosslinking reagents include, but are not limited to, glutaraldehyde, formaldehyde, biocompatible/bifunctional aldehydes, carbodiimides, hexamethylene diisocyanate, bis-imidates, polyglycerol polyglycidyl ether, glyoxal, and combinations thereof. Because different cross-linking agents form bonds of varying stability, the choice of cross-linking agent can affect the resorption rate and in vivo durability characteristic of the implant. For example, glutaraldehyde forms more stable crosslinks than formaldehyde or carbodiimide, and therefore glutaraldehyde is a preferred choice for producing an implant with increased in vivo durability, and a decreased resorption rate. On the other hand, if a highly resorbable implant is desired, then formaldehyde or carbodiimide are preferred cross-linking agents.

Intermolecular crosslinks can also be established through a dehydrothermal process (heat and vacuum) which results in peptide bond formation between an amino group of lysine or hydroxylysine and a carboxyl group of aspartic acid or glutamic acid. The crosslinked device has a relatively high thermal stability between about 55° C. to 85° C., preferably between 65° C. to 75° C., for sufficient in vivo stability. This can be achieved through manipulation of the crosslinking conditions, including reagent concentration, temperature, pH, and time.

One preferred method of manufacture begins with preparation of an acid dispersion of type I collagen fibers, which are coacervated with an alkaline solution such as an ammonium hydroxide or a sodium hydroxide solution. The coacervated fibers are partially dehydrated and molded into a predetermined size and shape of defined density. The molded fibers are then lyophilized, using well-known procedures for lyophilizing porous collagen-based matrices.

For an exemplary implant of the present invention, the matrix is lyophilized at −20° C. under a vacuum of less than 400 millitorr for about 48 hours, followed by drying under vacuum for about 12 to 24 hours at about 20° C. The lyophilized matrix is then cross-linked using a cross-linking agent commonly employed by medical implant manufacturers such as glutaraldehyde, formaldehyde or any other bifunctional agents that can react with amino, carboxyl, hydroxyl and guanidino groups of proteins and polysaccharides. Formaldehyde vapor is frequently used for cross-linking the porous collagen-based materials due to its volatility and therefore can be used for cross-linking the meniscus implant.

The mold used can be designed for a specific implant, for example, a meniscal augmention device used to repair segmental defects in the meniscus, an entire meniscal implant, or other desired shape depending on the nature of the implant being formed. The mold can also be designed to produce and/or form the matrix as a sheet or other shape and/or size designed for versatility, in that a surgeon can cut, roll or twist the sheet as desired to form a particularized shape for an individual implant. In a preferred embodiment, the matrix components are poured or shaped onto a suitable surface, and are not molded. Depending on the shape chosen, and the characteristics desired for the implant, the matrix may be molded to shape or cut to shape, and if cut may be cut prior to or after the coating step.

For meniscal implants, the mold used has a dimension similar to a human medial or lateral meniscus, but typically for a medial meniscus implant, the mold has a dimension of approximately 80% of an averaged human meniscus. This size is similar to a subtotal resection during partial meniscectomy procedure, leaving a 2 to 3 mm vascular peripheral meniscal rim intact for the attachment of the implant device and for the infiltration of host cells and nutrient into the scaffold matrix. For a lateral meniscus, the dimension of the mold is slightly modified to accommodate the anatomical difference between menisci. The shape can be defined by the shape and size of the defect which is to be repaired, or can define a shape and/or size larger than that of the defect, in which case the coated matrix can be cut to a desired shape and/or size that complements the defect.

Application of the teachings of the present invention to a specific problem or environment is within the capabilities of one having ordinary skill in the art in light of the teachings contained herein. Examples of the products and processes of the present invention appear in the following examples.

### EXAMPLE 1

Fabrication of Collagen Matrix

(A) The collagen content of highly purified type I collagen fibrils (e.g., prepared as described in Example 2 of U.S. Pat. No. 5,681,353) is determined either by gravimetric methods or by determining the hydroxyproline content assuming 13.5% by weight of hydroxyproline in Type I collagen. The amount of purified material needed to fabricate a given density of an implant is then determined and weighed.

(B) A solution of fibrillar collagen is fit into a mold of specified dimensions, and the collagen fibers are laid down in random manner or in an oriented manner. In the oriented manner, circumferential orientation of the fibers is produced by rotation of the piston about its principal axis as the material is compressed in the mold; radial orientation is produced by manual painting of the collagen fibers in a linear, radially directed fashion.

(C) The fibers are frozen at −20° C., turned out of the mold, and thawed at room temperature. The fibers are then resuspended in phosphate buffered saline, put back into the mold in the desired orientations(s), and compressed with the piston. The compressed fibers are then refrozen at −20° C., and then thawed at room temperature.

(D) The matrix is then cross-linked using any of the following alternative procedures: (a) The matrix is crosslinked by soaking in a 0.2% glutaraldehyde solution, pH 7.6 for 24 (±0.5) hours. The crosslinked matrix is subsequently rinsed repeatedly in 500 ml of phosphate buffered saline (PBS) solution, pH 7.4, for 4, 8, 24, and 48 hours; (b) The matrix structure is crosslinked in 5% polyglycerol polyglycidyl ether in 50% ethanol and 0.1M Na₂CO₃ at pH 10.0 for 24 (±2) hours. The crosslinked matrix is rinsed for 4, 8, 24 and 48 hours, each with 500 ml of PBS, pH 7.4; (c) The matrix structure is crosslinked in the presence of 10 ethyl-3-(3-dimethyl aminopropyl) carbodiimide (10 mg matrix) in 0.9% NaCl, pH 4.7 at room temperature for 24 (±2) hours. The addition of carbodiimide is made every 3-4 hours, and the pH is adjusted to 4.7 after each addition of carbodiimide; or (d) The matrix is crosslinked with formaldehyde vapor generated from a 2% HCHO solution at 22° C. for 5-24 hours. The crosslinked matrix is rinsed repeatedly in distilled water.

(E) The crosslinked and rinsed matrix is then lyophilized.

(F) Optionally, the matrix can be further crosslinked dehydrothermally by vacuum and heat. The vacuum is first applied to reduce the residual water content to a minimal level (some structural water, about 3%, may still be associated with collagen triple-helix as part of the structure stabilizing factor). The heat is increased in steps to 110° C. under vacuum for 24 (±2) hours.

Fabrication of Collagen-GAG Matrix

The procedure described above for fabrication of a collagen matrix is followed, except that step (B) is replaced with the following:

(B) The collagen material is dispersed in 0.01M HCl solution at pH 2-2.5. Predetermined amounts of various GAGs are weighed and dissolved in water. For example, for a given density of 0.25 g/cm³, the collagen content will be 0.244 g, the hyaluronic acid content will be 0.003 g, and the chondroitin sulfate content will be 0.003 g for a 2.5% GAG content. The GAG solution is mixed in with the collagen solution and placed in the mold in the desired orientation.

### EXAMPLE 2

Coating of the Matrices

A solution of sodium hyaluronate is brushed onto a matrix of Example 1, and exposed to formaldehyde vapor generated from a 2% HCHO solution at 22° C. for 5-10 hours to cross-link the coating to the matrix. The crosslinked and coated matrix is then rinsed repeatedly in distilled water.

### EXAMPLE 3

(A) Approximately 700 g of a Type I or Type II collagen dispersion (e.g., prepared as described in Example 6 or 8 of U.S. Pat. No. 5,681,353) is weighed into a 2 liter vacuum flask. Approximately 120 ml 0.6% ammonium hydroxide is added to the dispersion to coacervate the collagen. About 80 ml 20% NaCl is then added to the coacervated fibers to further reduce the solution imbibition between the fibers.

(B) The fully coacervated fibers are dehydrated to about 70 g to 80 g in a perforated mesh basket to remove the excess solution from the fibers. The partially dehydrated collagen fibers are inserted into a mold of specified dimension related to the dimensions of the defect to be remedied. Further dehydration is ongoing in the mold using a constant (between 300 grams to 700 grams) weight to slowly remove the water from the fibers, yet maintaining the same density throughout. This slow dehydration process lasts for about 24 hours until the desired dimension (about 8 mm in thickness) is reached. The dehydrated collagen matrix is further shaped to the desired form figure.

(C) The dehydrated collagen fibers are frozen at −20° C. for at least 4 hours before freeze drying in a Virtis freeze dryer. The frozen collagen fibers are first dried at −10° C. for 48 to 72 hours, followed by drying at 20° C. for 16 to 24 hours at a vacuum of 400 millibar.

(D) The freeze dried matrices are subjected to a formaldehyde crosslinking procedure. The matrices are crosslinked for 40 hours in a closed chamber of formaldehyde vapor generated from a 2% formaldehyde solution at 22° C. The crosslinked matrices are vented extensively to remove the non-bounded formaldehyde.

(E) A solution of coating material is then applied to the matrices.

(F) The coated matrices are then subjected to a heat and vacuum treatment to further crosslink the matrices, and to crosslink the coating material to the matrices.

(G) The matrices are cut to the shape of the segmental defect of the meniscus to be repaired. The cut matrices are extensively rinsed in pyrogen free distilled water to remove the residual salts and formaldehyde to the extent that the matrices are biocompatible in vitro and in vivo. The rinsed matrices are dried under a hepafilter and are packaged and sterilized.

### EXAMPLE 4

In vitro testing was performed to determine the ability of the implant to function and/or serve as a regeneration template for normal meniscal tissues.

Suture Pullout Test

The purpose of the suture pullout test is to ascertain that the suture pullout strength of the hydrated matrix exceeds the strength requirement for surgical implantation. The suture pullout test is determined by passing a 2-0 polyester suture (Davis & Geck, Danbury, Conn.), attached to a needle, through the product 2 mm from the outer edge of a pre-hydrated sample. A knot is tied and the loop is attached to the hook of a load cell on a Chatillon mechanical tester, TCD 200 (AMETEK Test and Calibration Instruments Division, Largo, Fla.). The sample is pulled by the cross-head at a speed of 1 inch per minute until the suture pulls out. The maximum force to failure is the suture pullout value. A suture pullout force greater than 2 lbs. meets the strength requirement for surgical implantation.

Density Test

The purpose of the density test is to ensure that the density is within the design guidelines for pore structure for tissue ingrowth. The dimensions of a matrix are first measured with a caliper to within 0.2 mm. The volume is then calculated. The matrix is then dried in an oven for 4 hours at 100° C. The dry matrix is weighed to within 0.2 mg accuracy and the density is calculated in g/cm³. The matrix has an average density of 0.20 g matrix/cm³.

Pore Size Determination

The purpose of the pore size determination is to ensure that the pores of the matrix are in a range that permits tissue ingrowth. Scanning Electron Micrographs are taken of the matrix at magnifications of 20×, 40×, and 100×. ImageJ software (National Institutes of Health, available on the Internet (http://) at rsb.info.nih.gov/nih-image/index.html) is used to perform a pore size analysis based on the pixel count of the pores. A representative sample of the SEM image of the matrix is analyzed and the pore sizes are counted into preset bins in accordance with the pore size. One sample matrix has a pore size of 50 to 500 micrometers.

Compressive Shear Strength Test

The compressive shear strength test is used to evaluate the relative compressive shear strength between two meniscus implants, for example an uncoated implant and a coated implant. An apparatus was designed to hold the implants against a smooth polyethylene wheel that is oscillated with a bell crank operated by an electric motor. Samples are mounted with adhesives onto a small block of wood such that the distance from the edge of outer rim to outer rim (of the implants) is 30 mm. This distance allows the 2 inch diameter wheel to match the profile of the two contoured pieces of each sample. A platform above the mounted implant holds stacked weights, which apply pressure holding the mounted implant against the wheel, to mimic the weight on a loaded joint. The tester is mounted on the wall of a container such that the wheel and mounted implant are immersible in water.

Segments of a dry implant are mounted to a wooden block with rubber cement (Duco Cement, ITW Devcon, Danvers, Mass.) affixing the bottom surface of the specimen to the block. The samples are allowed to dry for about 24 hours. The mounted implants are placed under a pressure of 5 psi and in room temperature water where the superior surface of the implant is held against the oscillating wheel. The oscillation of the wheel is held constant at 12 cycles/min. For each sample tested, the mean time to failure (e.g., cracking or detachment of the implant) is measured. Samples with greater lubricity can withstand the compressive shear stresses longer under the above test conditions.

### EXAMPLE 5

Surgical Implantation Technique And Cadaveric Testing

A meniscal augmentation device comprising a Type I collagen-GAG matrix is formed according to the preceding Examples and is evaluated in human cadaveric implantations. The devices are implanted into both horizontal cleavage tears and segmental defects. All knees are approached by standard arthroscopic portals. For medial meniscal tears, the arthroscope is placed in the mid lateral patella portal and instrumentation through the anterior medial portal. While preserving the stable portions of the meniscus, the torn and frayed portions of the meniscus are removed with arthroscopic biters and shavers.

A calibrated probe is then placed along the meniscal defect to obtain measurements of the defect. The probe is then laid upon the meniscal augmentation device and the device trimmed to match the defect. The trimmed portion of the meniscal augmentation device is then grasped with a specially modified arthroscopic grasper and inserted into the segment. While still within the grasper, the device is sutured in place by passing ten inch 2-0 PDS sutures down the bore of the grasper, through the meniscal augmentation device and then through the native meniscus. The sutures are tied directly over the capsule beneath the skin. Additional sutures are placed as necessary to further secure the implant to the meniscal rim.

The knees are flexed and extended through the full range of motion. Despite significant swelling of the device, molding uniformly occurs to match the shape of the opposing articular cartilage and femoral condyle. Sequential 3D MRI images are obtained in several knees documenting stable placement of the implant, and appropriate excursion on the tibial plateau from 0 degrees to 120 degrees of flexion.

The foregoing disclosure of the preferred embodiments of the present invention has been presented for purposes of illustration and description. It is not intended to be exhaustive or to limit the invention to the precise forms disclosed. Many variations and modifications of the embodiments described herein will be apparent to one of ordinary skill in the art, and the scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be and still remain within the spirit and scope of the present invention.

All of the patents, publications and references mentioned herein are incorporated by reference in their entirety.

What is claimed is:

1. A biocompatible implant for use in the skeletomuscular system comprising:
a polymeric matrix that is at least partially bioresorbable, and a bioresorbable lubricious coating disposed on a surface of the polymeric matrix.

2. The implant of claim 1, wherein the implant is a cartilage implant, a ligament implant, a tendon implant, an interpositional joint spacer, a spinal disc implant, a meniscus implant, or a bone implant.

3. The implant of claim 1, wherein the implant is a meniscus implant, and wherein the bioresorbable lubricious coating is disposed on an articulating surface of the implant.

4. The implant of claim 1, wherein the polymeric matrix is a biopolymer-based matrix.

5. The implant of claim 4, wherein the biopolymer-based matrix is a collagen-based matrix.

6. The implant of claim 5, wherein the collagen-based matrix is a type I collagen-based matrix.

7. The implant of claim 5, wherein the polymeric matrix comprises, by dry weight, about 65-99 percent collagen fibers and about 1-35 percent glycosaminoglycans.

8. The implant of claim 7, wherein the glycosaminoglycans are selected from the group consisting of chondroitin 4-sulfate, chondroitin 6-sulfate, keratan sulfate, dermatan sulfate, heparin, heparan sulfate, hyaluronic acid, and combinations thereof.

9. The implant of claim 1, wherein the coating is a discrete coating.

10. The implant of claim 1, wherein the coating is an integrated coating.

11. The implant of claim 10, wherein the integrated coating is crosslinked to the polymeric matrix.

12. The implant of claim 10, wherein the integrated coating comprises sodium hyaluronate crosslinked to the polymeric matrix.

13. The implant of claim 1, wherein the coating is selected from the group consisting of chondroitin 4-sulfate, chondroitin 6-sulfate, keratan sulfate, dermatan sulfate, heparin, heparan sulfate, hyaluronic acid, and combinations thereof.

14. The implant of claim 13, wherein the coating is sodium hyaluronate.

15. The implant of claim 1, wherein the coating is chondroitin sulfate.

16. The implant of claim 1, wherein the coating is type I collagen.

17. A meniscus implant comprising:
a polymeric matrix, and
a lubricious coating disposed on at least one articulating surface of the polymeric matrix.

18. The implant of claim 17, wherein the meniscus implant is a prosthetic meniscus.

19. The implant of claim 17, wherein the meniscus implant is a meniscal augmentation device.

20. The implant of claim 17, wherein said at least one articulating surface comprises the surface of the matrix that will face the femoral condyles upon implantation.

21. The implant of claim 20, wherein said at least one articulating surface further comprises the surface of the matrix that will face the tibial plateau upon implantation.

22. The implant of claim 17, wherein the polymeric matrix is at least partially bioresorbable.

23. A method of fabricating an implant, comprising:
providing a plurality of biocompatible and bioresorbable fibers in a mold;
cross-linking the biocompatible and bioresorbable fibers to produce a matrix; and
applying a coating material to the surface of the matrix.

24. The method of claim 23, further comprising lyophilizing the matrix prior to said application step.

25. The method of claim 23, wherein said application step is performed prior to said cross-linking step.

26. The method of claim 23, further comprising dehydrothermally cross-linking the coating material to the matrix after said application step.

27. A method of fabricating an implant, comprising:
providing a plurality of biocompatible and bioresorbable fibers in a mold;
lyophilizing the molded fibers to produce a dry and porous matrix;
contacting the dry and porous matrix with a cross-linking agent; and
applying a coating material to the surface of the matrix.

28. The method of claim 27, further comprising dehydrothermally cross-linking the coating material to the matrix after said application step.
